(19) United States
(12) Patent Application Publication
(10) Pub. No.: US 2003/0087260 A1
(43) Pub. Date: May 8, 2003

(54) Family-based association tests for quantitative traits using pooled DNA

(76) Inventors: Joel S. Bader, Stamford, CT (US); Pak Sham, London (GB)

Correspondence Address: MINTZ, LEVIN, COHN, FERRIS, GLOVSKY AND POPEO, P.C, One Financial Center, Boston, MA, 02111, US

(21) Appl. No.: 10/139,850

(22) Filed: May 7, 2002

Related U.S. Application Data
(60) Provisional application No. 60/289,068, filed on May 7, 2001.

(51) Int. Cl. C12Q 1/68; G06F 19/00; G01N 33/48; G01N 33/50

(52) U.S. Cl. 435/6; 702/20

(57) ABSTRACT

While SNP-based marker sets and population-level DNA repositories are approaching sufficient size for whole-genome association studies, individual genotyping remains very costly. Pooled DNA tests are a less costly alternative, but uncertainty about loss of power due to allele frequency measurement error and population stratification hinder their use. Here we describe how to optimize pooled tests as an explicit function of measurement error, and we present family-based tests that eliminate stratification effects. We show that identification of functional genetic variants and linked markers may be feasible with current-day instruments.

### RELATED APPLICATIONS

This application claims priority to U.S. Ser. No. 60/289,068, filed May 7, 2001 which is incorporated herein by reference in its entirety.

### INTRODUCTION

Association tests of outbred populations are thought to have greater power than traditional family-based linkage analysis to identify the genetic variants contributing to complex human diseases (Risch and Merikangas, 1996; Ott 1999; Ardlie 2002). A genome scan based on allelic association would require approximately 100,000 markers, estimated by dividing the 3.3 gigabase human genome by the several kilobase extent of population-level linkage disequilibrium (Abecasis et al 2001; Reich et al. 2001). Single-nucleotide polymorphisms (SNPs) occur at sufficient density to provide a suitable marker set (Collins et al. 1997). Furthermore, SNPs in coding and regulatory regions have additional value as potential functional variants.

Individual genotyping remains prohibitively expensive for a genome scan. One method to reduce cost is to pool DNA from individuals with extreme phenotypic values and to measure the allele frequency difference between pools (Barcellos et al.,1997; Daniels et al., 1998; Fisher et al., 1999; Hill et al., 1999; Shaw et al., 1998; Stockton et al., 1998; Suzuki et al., 1998). Initial attention focused on pooled designs for dichotomous traits and case-control studies (Risch and Teng 1998). More recently, pooled tests have been discussed for quantitative traits, a more appropriate model for diseases such as obesity and hypertension. In the absence of experimental error, the optimal design for an unrelated population is to compare frequencies between pools of the most extreme 27% of individuals ranked by phenotypic value, retaining 80% of the information of individual genotyping (Bader et al., 2001). Experimental sources of error, primarily allele frequency measurement error, degrade the test power (Jawaid et al., 2002).

Population stratification poses a second challenge to practical use of pooled tests for human populations. Genomic control methods, developed to reduce stratification effects in genotype-based association tests (Devlin and Roeder 1999; Pritchard and Rosenberg 1999; Pritchard et al. 2001; Zhang and Zhou, 2001), are not directly applicable to pooled tests.

Here we present optimized pooled DNA test designs, including family-based tests robust to stratification. Estimates of test power explicitly include allele frequency measurement error. This distinguishes our treatment from prior theoretical work, permits the optimization of test design as a function of known parameters, and provides a bridge to experimentalists seeking practical guidance for whether to attempt and how to perform pooled association tests.

### SUMMARY OF THE INVENTION

The invention is drawn to a method for detecting an association in a population of unrelated individuals between a genetic locus and a quantitative phenotype, wherein two or more alleles occur at the locus, and wherein the phenotype is expressed using a numerical phenotypic value whose range falls within a first numerical limit and a second numerical limit. This method comprises the steps of:

a) obtaining the phenotypic value for each individual in the population;

b) determining the minimum number of individuals from the population required for detecting the association using a non-centrality parameter;

c) selecting a first subpopulation of individuals having phenotypic values that are higher than a predetermined lower limit and pooling DNA from the individuals in the first subpopulation to provide an upper pool;

d) selecting a second subpopulation of individuals having phenotypic values that are lower than a predetermined upper limit and pooling DNA from the individuals in the second subpopulation to provide a lower pool;

e) for one or more genetic loci, measuring the frequency of occurrence of each allele at said locus in the upper pool and the lower pool;

f) for a particular genetic locus, measuring the difference in frequency of occurrence of a specified allele between the upper pool and the lower pool; and

g) determining that an association exists if the allele frequency difference between the pools is larger than a predetermined value.

In one embodiment of the invention, the difference in frequency of occurrence of the specified allele has associated with it an error of measurement. In one aspect of the invention the error of measurement is 0.04. In another, the error of measurement is 0.01.

In another embodiment of the invention, the predetermined lower limit is set so that the upper pool ranges from including the highest 37% of the population to including the highest 19% of the population and the predetermined upper limit is set so that the lower pool ranges from including the lowest 37% of the population to including the lowest 19% of the population. In another aspect of the invention, the predetermined lower limit is set so that the upper pool includes the highest 27% of the population and the predetermined upper limit is set so that the lower pool includes the lowest 27% of the population.

In another embodiment of the invention, the genetic locus has two alleles.

In another embodiment of the invention, the population includes individuals who may be classified into classes. In one aspect of the invention, the classes are based on an age group, gender, race or ethnic origin. In another aspect of the invention, the members of a class are included in the pools.

In another embodiment of the invention the method is used for determining the genetic basis of disease predisposition.

In another embodiment of the invention, the genetic locus which is analyzed for determining the genetic basis of disease predisposition contains a single nucleotide polymorphism.

### BRIEF DESCRIPTION OF THE DRAWINGS

FIG. 1. The information retained by the between-family pooled test design, expressed as a fraction of the information from individual genotyping followed by a between-family test, is depicted sibships of size 4, 2, and 1, each population having 1000 total individuals. The optimal pooling fraction, indicated by an arrow, shifts to lower values as the number of sibs per family decreases. The optimal fraction and corresponding information retained also shift to lower values as the minor allele frequency decreases, with results shown for frequencies 0.1 and 0.01. The raw measurement error is 0.01.

FIG. 2. The optimal number of sibs to select from each family (top panel) and the information retained relative to individual genotyping (bottom panel) are shown for sibship sizes 2-5, 6, 8, 16, and 32 as a function of the scaled measurement error κ. For sibships through 5, it is always optimal to select just the highest and lowest sib.

FIG. 3. The optimal fraction of families to select (top panel) and information retained (lower panel) are displayed for sibships of size 2 through 6 as a function of the scaled measurement error κ.

FIG. 4. The optimal pooling fraction (top panel) and information retained (bottom panel) for between-family and within-family tests of a population of 500 sib-pairs are shown as a function of raw measurement error for marker frequencies 0.5 and 0.01. The within-family tests include pre-selection of discordant-like families.

FIG. 5. The optimal pooling fraction (top panel) and the information retained (bottom panel) from exact numerical calculations (solid line) and an analytical fit (dashed line) are displayed as a function of the normalized measurement error κ. The fit coincides with the exact results for the information retained.

### DETAILED DESCRIPTION

We present optimized designs for pooled DNA tests conducted on a population of N/s families, each a sibship of size s (N total individuals). The genotypic correlation within a sibship is denoted r, with typical values of ¼, ½, and 1 for half-sibs, full-sibs, and monozygotic twins. Sibships may also represent inbred lines; in this case, r is the genetic correlation within each line. Sibs in different families are assumed to have uncorrelated genotypes.

To conduct a pooled DNA test for association of a particular allele A₁ with a quantitative trait, individuals are selected for an upper pool, comprising higher phenotypic values, and a lower pool, comprising lower phenotypic value, using designs reminiscent of selection strategies for optimizing breeding value and for QTL mapping (Hill 1971; Kimura and Crow 1978; Ollivier et al. 1997). We restrict attention to balanced designs in which each pool has ƒN individuals, with ƒ≦0.5 defined as the pooling fraction. Balanced designs are favored when high and low phenotypes are treated symmetrically; asymmetry can favor unbalanced designs (Jawaid et al., 2002).

We consider four designs: (i) unrelated individuals (s=1), in which the ƒN individuals having highest and lowest phenotypic values are selected for the upper and lower pools respectively; (ii) between-family, in which all s sibs from the ƒN/s families having highest and lowest mean phenotypic values are selected for the upper and lower pools; (iii) within-family, in which the s′ sibs having highest and lowest phenotypic values within each family are selected for the upper and lower pools, yielding a pooling fraction ƒ=s′/s; (iv) within-family with pre-selection of discordant families, in which a fraction ƒ′ of families with greatest within-family phenotypic variance are selected,

$$Var = {\sum }_{s} {( {X}_{s} - \overline{X} )}^{2}$$

where X₅ is the phenotype of sib s and \overline{X} is the family mean, then the extreme high and low sib within each selected family are selected for the upper and lower pool for a final pooling fraction ƒ=ƒ′/N.

A suitable statistic for a two-sided test for each design is

$${Z}^{2} = \frac{{( {\hat{p}}_{U} - {\hat{p}}_{L} )}^{2}}{Var ( {\hat{p}}_{U} - {\hat{p}}_{L} )} ,$$

where the estimated frequencies of allele A₁ in the upper and lower pools are denoted \hat{p}ᵤ and \hat{p}_{L}. The variance is the sum of three terms, Var(\hat{p}_{U}−\hat{p}_{L})=V_{S}+V_{C}+V_{M}. The sampling variance V_{S} represents the unavoidable error in estimating the population frequency from a finite sample. The concentration variance V_{C} arises from sample-to-sample DNA concentration variance within a pool. The measurement variance is V_{M}=2ε², where ε is the experimental allele frequency measurement error for each pool. We assume that the three sources of variation are independent, which should be justified when individual and pooled DNA samples are treated uniformly. In an ideal experiment, V_{C} and V_{M} vanish, and the total variance is V_{S}.

Under the null hypothesis, Z² has a χ² distribution with one degree of freedom. Under the alternate hypothesis, the tested marker is assumed to be a bi-allelic quantitative trait locus (QTL) with alleles A₁ and A₂ occurring at frequencies p and (1−p)≡q. For between-family tests, the alleles are assumed to be in Hardy-Weinberg equilibrium and the population is assumed to be random mating; these assumptions may be relaxed for within-family tests. The variance of the allele frequency per individual is σₚ²=pq/2. For each design, the allele frequency is estimated as \hat{p}=(\hat{p}_{U}+\hat{p}_{L})/2. The estimated variance of the allele frequency per individual is denoted \overline{σ}ₚ² and equals \hat{p}(1−\hat{p})/2.

The mean phenotypic effects are m_{G}=a, d, and −a for genotypes G=A₁A₁, A₁A₂, and A₂A₂, respectively. The dominance ratio d/a describes the inheritance mode with typical values −1, 0, and 1 for pure recessive, additive, or dominant inheritance. The proportion of trait variance accounted for by the QTL is denoted σ_{Q}²,

σ_{Q}²=2pq[a−d(p−q)]²+[2pqd]²=σ_{A}²+σ_{D}².

The mean QTL effect is m=(p−q)a+2pqd. Phenotypic values are assumed to be normally distributed for each genotype with mean μ_{G}=m_{G}−m and residual variance σ_{R}²=1−σ_{Q}² arising from all genetic and environmental factors other than the QTL. The distribution of phenotypic values in the population is a mixture of three normal distributions with overall mean 0 and variance 1. The total phenotypic correlation between sibs from genetic factors (including the QTL) and environmental factors is termed t.

1

The non-centrality parameter (NCP),

NCP=[E(\hat{p}_{U}−\hat{p}_{L})]²/Var(\hat{p}_{U}−\hat{p}_{L}),

measures the information provided by a pooled DNA test. The notation E(Ô) is the expectation of an observable Ô. The approach followed below is to evaluate the numerator of the NCP as a function of the model parameters, providing accurate analytical results when possible and simulation results otherwise. For the denominator of the NCP, analytical results are obtained for the null hypothesis. For the alternative hypothesis, the expected allele frequencies for each pool have offsetting changes from p to p±δp (see Methods for derivation), and the value of the denominator decreases by a small value proportional to (δp/p)². We make a conservative approximation by ignoring the change and using the null hypothesis denominator for the alternative hypothesis as well. In this case, the NCP equals (z_{α/2}−z_{1-β})², where α and β are the type I and II error rates for the two-sided test. Maximizing the NCP optimizes the test.

The denominator of the NCP is shown in the Methods to have the form

$$\begin{matrix}{V}_{S} + {V}_{C} + {V}_{M} = \frac{2 G {\hat{σ}}_{p}^{2}}{Nf} + \frac{2 {τ}^{2} {\hat{σ}}_{p}^{2}}{Nf} + 2 {ϵ}^{2} \\ = \frac{2 {\hat{σ}}_{p}^{2}}{Nf} \cdot ( G + {τ}^{2} ) \cdot [ 1 + \frac{Nf {ϵ}^{2}}{( G + {τ}^{2} ) {\hat{σ}}_{p}^{2}} ] \\ = \frac{2 {\hat{σ}}_{p}^{2}}{Nf} \cdot ( G + {τ}^{2} ) \cdot ( 1 + f {κ}^{2} )\end{matrix}$$

where τ is the coefficient of variation for DNA concentration. The constant G depends only on the family structure and equals 1 for pools of unrelated individuals, sR for the between-family design, and (1−r) for both within-family designs; the standard notation R relates the sib genotypic correlation r to family-based variance components,

$$R = \frac{1}{s} \cdot [ 1 + ( s - 1 ) r ] .$$

Typically τ is less than 10%; τ² may usually be ignored relative to G. The term κ² is used as shorthand,

κ²≡ε²/[(G+τ²)\hat{σ}ₚ²/N].

Referred to as the measurement error, κ represents the raw measurement error, ε, scaled by the remaining sources of error in the allele frequency difference. In practice, κ can be calculated prior to pooling because it depends on known quantities.

The numerator of the NCP is shown in the Methods to have the form

$${[ E ( {\hat{p}}_{U} - {\hat{p}}_{L} ) ]}^{2} = \frac{4 {σ}_{A}^{2} {\hat{σ}}_{p}^{2} {φ [ {Φ}^{- 1} ( 1 - f ) ]}^{2}}{{σ}_{R}^{2} {f}^{2}} \cdot F$$

where φ(z) is the normal density (2π)^{-½} exp(−z²/2), Φ(z) is the cumulative normal probability and Φ⁻¹(z) its functional inverse. The constant F equals 1 for pools of unrelated individuals, R²/T for between-family pools, and (1−R)²/(1−T) for within-family pools without pre-selection. For the within-family design using discordant-like pre-selection, F=(1−r)²/2(1−t) for sib-pairs (expressions for larger sibships are unwieldy). The term R has the same definition as before, and T is the standard factor relating the sib phenotypic correlation t to family-based variance components,

$$T = \frac{1}{s} \cdot [ 1 + ( s - 1 ) t ] .$$

Combining terms, the analytical result for the NCP, valid for small QTL effect, is

$$NCP = \frac{N {σ}_{A}^{2}}{{σ}_{R}^{2}} \cdot \frac{F}{G + {τ}^{2}} \cdot \frac{ 2 {φ [ {Φ}^{- 1} ( 1 - f ) ]}^{2}}{f + {f}^{2} {κ}^{2}} .$$

The first factor is identical to the NCP for an association test performed by individual genotyping on a population of N unrelated individuals; the second factor, with τ=0, is the correction for individual genotyping a population of N/s families each having s sibs and then performing either a between-family test, with FIG =R/sT, or a within-family test, with F/G=(s−1)R/s(1−T). The third factor represents the fraction of information retained when the association test is performed by pooling instead of individual genotyping, and maximizing this factor with respect to the pooling fraction ƒ provides the optimal pool size. When the measurement error ε=0, tests are optimized with ƒ=0.27 and 80% of the information is retained (Bader et al. 2001). As ε increases, the maximum information that can be retained is determined entirely by the single collective term κ.

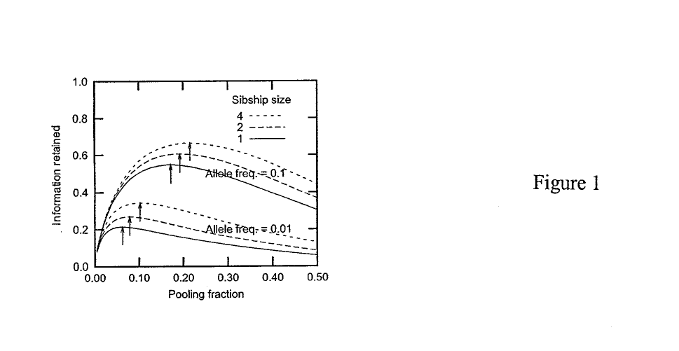

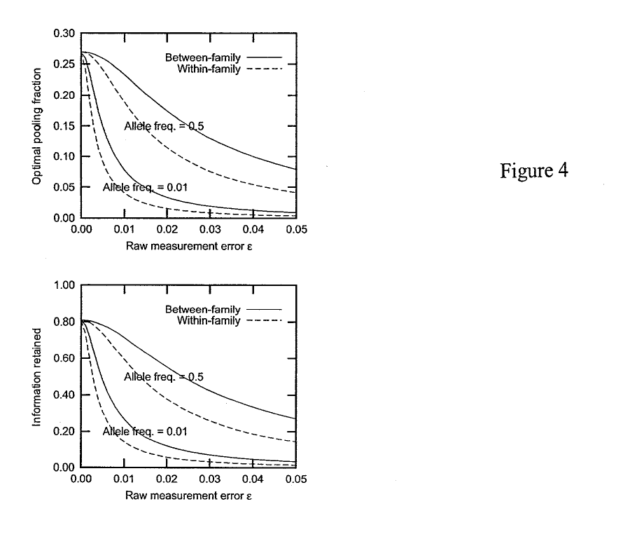

Expressions for F, G, and κ² are summarized in Table I, and we now provide examples of each family-based design. Information retained by the between-family design is depicted in FIG. 1, with results for 3 sibship sizes: sib-quads, sib-pairs, and unrelated individuals, each population having 1000 total individuals. The optimal pooling fraction, indicated by an arrow, shifts to lower values as the number of sibs per family decreases. The optimal fraction and corresponding information retained also shift to lower values as the minor allele frequency decreases, with results shown for frequencies 0.1 and 0.01. The raw measurement error ε=0.01 in this example, and the pooling fraction and information retained would decrease for larger ε (see FIG. 4 for examples of changing ε).

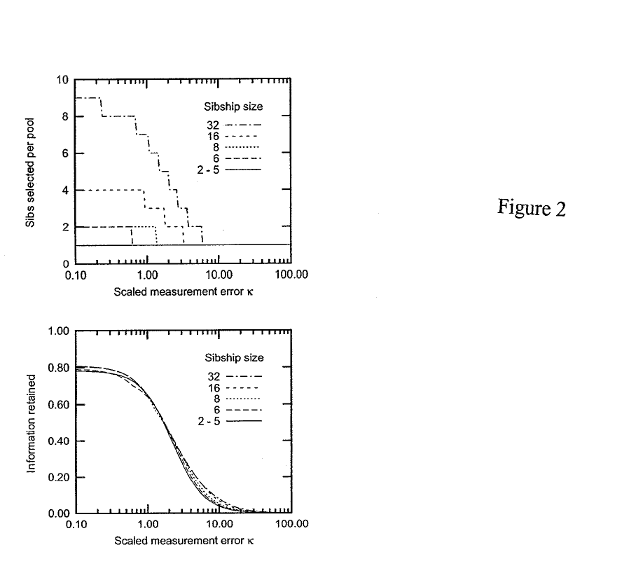

In FIG. 2, the optimal number of sibs to select from each family (top panel) and the information retained relative to individual genotyping (bottom panel) are shown as a function of the scaled measurement error κ for sibship sizes of 2-5, 6, 8, 16, and 32. For sibships through 5, it is always optimal to select just the highest and lowest sib. For larger families and small measurement error, the top and bottom quarters of the sibs are pooled and 80% of the information is retained. The pooling fraction and information decrease as the measurement error increase.

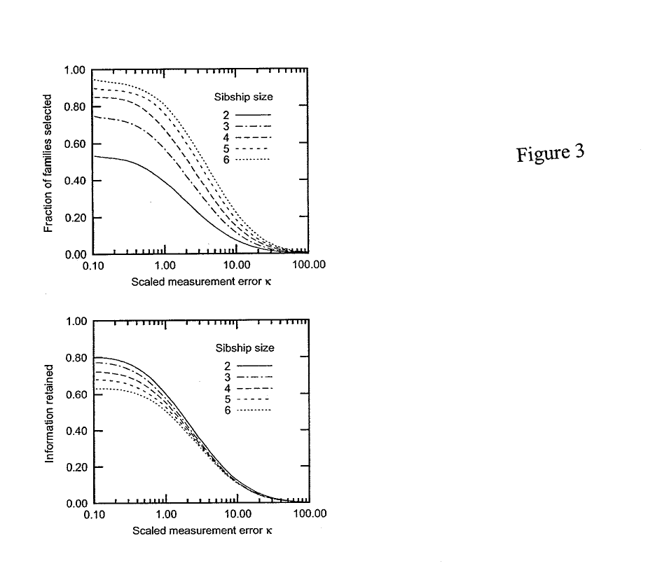

Within-family tests can be improved by pre-selection of discordant-like families, as shown in FIG. 3. The optimal fraction of families to select (top panel) and information retained (bottom panel) are displayed for sibships of size 2 through 6 as a function of the scaled measurement error κ (results determined by computer simulation). The fraction of families and information retained both decrease as κ increases. Discordant pre-selection has the greatest benefit for sib-pairs: for the smallest values of κ, only 56% of families are selected, retaining 80% of the information; had all families been used, only 60% of the information would have been retained. Pre-selection is less important for trios and larger sibships.

In FIG. 4, the optimal pooling fraction (top panel) and information retained (bottom panel) using between-family pools and using within-family pools with discordant-like pre-selection are displayed for a population of 500 sib-pairs (1000 individuals) as a function of the raw measurement error ε. Results are shown marker frequencies 0.5 and 0.01. With no measurement error, the optimal pooling fraction of 0.27 retains 80% of the information in each case. As measurement error increases, the optimal pooling fraction decreases, as does the information retained.

The information loss increases for rarer alleles and is worse for the within-family test than for the between-family test. This behavior can be deduced from the scaled error κ², which is inversely proportional to the allele frequency sampling variance. Since the sampling variance is 3× smaller within-family vs. between-family, κ² is 3× larger, 4Nε²/p(1−p) vs. 4Nε²/3p(1−p), and more information is lost. The inverse dependence of κ² on the allele frequency explains the decrease in power for rare alleles.

Because the allele frequency difference between sibs is uncorrelated from their allele frequency mean, the between-family and within-family tests are independent estimators of σ_{A} even when individuals contribute their DNA under both designs. The NCP of a combined test is the sum of the NCPs for each test and it too follows a χ² distribution with 1 degree of freedom. In practice, estimates for σ_{A} may obtained by inverting the expressions for E(\hat{p}_{U}−\hat{p}_{L}) provided in Table I, then weighting each estimator by the inverse of its variance.

Population stratification may be indicated by a difference between the estimates for σ_{A} from a between-family and within-family test. In the absence of stratification, the difference follows a normal distribution with variance

Var[\hat{σ}_{A+}−\hat{σ}_{A-}]=V₊·[ƒ₊²Tσ_{R}²/4y₊²R²\hat{σ}ₚ²]+V₋·[ƒ₋²(1−T)σ_{R}²/4y₋²(1−R)²\hat{σ}ₚ²]

where the “+” and “−” subscripts refers to the between-family and within-family designs respectively, y_{±}=φ[Φ⁻¹(1−ƒ_{±})], and V represents the total variance, V_{S}+V_{C}+V_{M}, for each design. When stratification is indicated, the between-family estimate of σ_{A} may be unreliable but the within-family estimate remains robust.

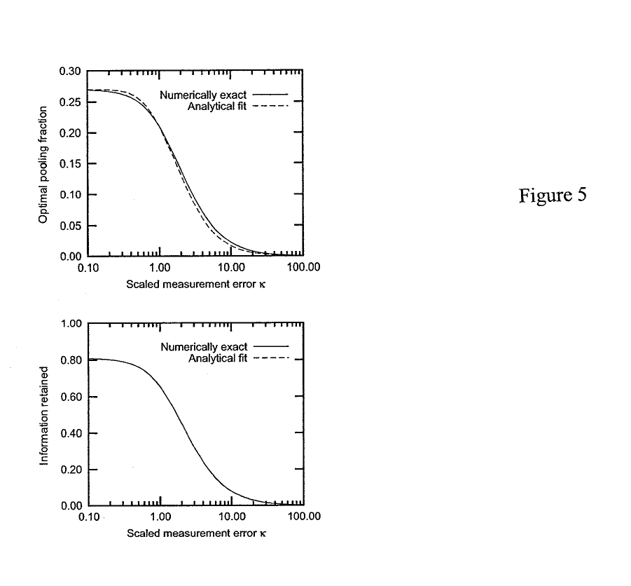

In FIG. 5, the optimal pooling fraction (top panel) and the information retained (bottom panel) are displayed as a function of the scaled measurement error κ. The information retained is calculated assuming no concentration variance. In addition to the numerically calculated results, an accurate fit is shown using the functional form

ƒ=1−Φ[A−(3/A)ln A−0.067],

with

$$A ( κ ) = \sqrt{2 + ln ( 1 + 3 {κ}^{2} + \frac{2}{π} {κ}^{4} )} .$$

A justification for this functional form is provided in the Methods. The greatest deviations for the pooling fraction are at κ=0.5, where the fit yields a pooling fraction that is 0.006 too high, and at κ=3.5, where the fit is 0.01 too low. The information retained using the analytical value for the pooling fraction coincides with the exact numerical results on the scale of the figure. The experimental measurement error ε corresponding to the scaled error κ depends on the population structure and marker frequency. For example, for a population of 500 cases, 500 matched unrelated controls, and 10% marker frequency, ε=0.0067 κ is the raw error corresponding to κ.

Based on the pooled designs described above, we outline a prospective study using 100,000 markers to detect QTLs with a 1% effect. If 100 false-positives are permitted from pooled tests (the false-positives may be resolved using individual genotyping) and 80% power is required, the NCP is 17. We assume pooling of discordant sib-pairs to protect against stratification effects. At the scaled error κ=1 where the pooled tests are still close to maximum power, the pooling fraction would be 21%, 65% of the information of a population would be retained, and a population of 2600 individuals would be required. The raw measurement error corresponding to κ=1 for this population size is 0.005 for an allele with 50% frequency and 0.002 for an allele with 5% frequency, 5× to 10× more precise than achieved by current-day instrumentation.

We can account for current-day precision by setting κ=10, which from FIG. 5 is seen to retain 7.7% of the information and corresponds to a pooling fraction of 1.6% of a total population of 22,000. In this case, the precision required for a pooled test is 0.017 for an allele with 50% frequency and 0.007 for an allele with 5% frequency. These precisions are within the range of current performance, especially if repeated measures are used to decrease the effective measurement error. The cost to collect and score such a population for multiple disease-related phenotypes would be under $50 million. Selection schemes could then be applied to generate pools for each phenotype in turn.

As noted previously, pooled tests perform worse for within-family tests and rare alleles, and may therefore be difficult to apply to disease-risk variants under negative selection pressure. The loss of power may be less severe for pharmacogenetic studies of variants affecting drug response, where selection pressure is absent, and for test crosses of model organisms (Grupe et al. 2001) or agricultural species whose marker frequencies are under experimental control.

The analysis provided here for quantitative traits may be extended to threshold characters yielding dichotomous classifications of a population. For case-control classification, the disease prevalence corresponds to the pooling fraction ƒ. When the quantitative character is available for measurement, it is approximately 4× more efficient to compare unrelated individuals with extremely high vs. extremely low characters than to compare the derived cases vs. controls (Bader et al. 2001).

In summary, we have derived the optimal pooling fractions for within-family and between-family tests of association. With ideal instrumentation, 80% of the information is retained and the optimal pooling fraction is 27%. As allele frequency measurement error increases, the optimal pooling fraction and the information retained both decreases. The information loss is more severe for low-frequency alleles and for within-family tests. The optimal pooling fraction depends on a single parameter representing the measurement error, and optimized pooling designs are provided as a function of this parameter.

### EXAMPLES

### Example 1

### Sampling Variance and Concentration Variance

Let pᵢ represent the frequency of allele A₁ for individual i, either 0, ½, or 1, and cᵢ represent the concentration of DNA contributed by this individual to a pool of n individuals. Neglecting measurement error, the allele frequency p* for the pool is

$$\begin{matrix}{p}^{*} = \sum \frac{{c}_{i} {p}_{i}}{\sum {c}_{j}} = p + \sum \frac{( {c}_{0} + δ {c}_{i} ) δ {p}_{i}}{\sum {c}_{0} + \sum δ {c}_{j}} \\ = p + \sum \frac{( \frac{1}{n} + \frac{δ {c}_{i}}{{nc}_{0}} ) δ {p}_{i}}{1 + \frac{1}{{nc}_{0}} \sum δ {c}_{j}} \\ \approx p + \sum δ {p}_{i} ( \frac{1}{n} + \frac{1}{n} \frac{δ {c}_{i}}{{c}_{0}} ) ( 1 - \frac{1}{n} \sum \frac{δ {c}_{j}}{{c}_{0}} ) \\ \approx p + \frac{1}{n} \sum δ {p}_{i} + \sum δ {p}_{i} ( \frac{δ {c}_{i}}{{nc}_{0}} - \sum \frac{δ {c}_{j}}{{n}^{2} {c}_{0}} ) \\ ≡ p + \frac{1}{n} \sum δ {p}_{i} + \frac{1}{n} \sum δ {p}_{i} δ {c}_{i}^{′}\end{matrix}$$

which defines the relative concentration error δcᵢ′. The terms δpᵢ and δcᵢ′ are uncorrelated, and each has expectation zero. Furthermore, the sum of the δcᵢ′ terms is constrained to be zero. The variance of p* is

$$\begin{matrix}Var ( {p}^{*} ) = \frac{1}{{n}^{2}} {\sum }_{i , j} Cov ( δ {p}_{i} , δ {p}_{j} ) + \frac{1}{{n}^{2}} {\sum }_{i , j} Cov ( δ {c}_{i}^{′} , δ {c}_{j}^{′} ) \\ Cov ( δ {p}_{i} , δ {p}_{j} ) \\ = \frac{1}{{n}^{2}} {\sum }_{i , j} {r}_{ij} {σ}_{p}^{2} + \frac{{τ}^{2}}{n} {σ}_{p}^{2}\end{matrix}$$

$$We have used$$

$$Cov ( δ {p}_{i} , δ {p}_{j} ) = \frac{p ( 1 - p )}{2} {r}_{ij} = {σ}_{p}^{2} {r}_{ij} and$$

$$Cov ( δ {c}_{i}^{′} , δ {c}_{j}^{′} ) = {τ}^{2} ( {δ}_{ij} - \frac{1}{n} ) \approx {τ}^{2} {δ}_{ij} ,$$

with the concentration coefficient of variation defined as τ≡[Var(cᵢ)]^{½}/c₀ and the genotypic correlation between a pair of individuals defined as rᵢⱼ.

For the between-family design, a pool of n individuals contains n/s sibships of size s and genotypic correlation r. The result for Var(p*) is

$$Var ( {p}^{*} ) = \frac{sR}{n} {σ}_{p}^{2} + \frac{{τ}^{2}}{n} {σ}_{p}^{2} ,$$

with R=(1/s)[1+(s−1)r]. Since the individuals in the upper and lower pools are unrelated, Vₛ+V_{C}=2 Var(p*).

For a within-family design, the allele frequency difference between pools is

$$Δ {p}^{*} = \frac{1}{n} {\sum }_{i} ( 1 + δ {c}_{i}^{′} ) δ {p}_{i} - \frac{1}{n} {\sum }_{j} ( 1 + δ {c}_{j}^{′} ) δ {p}_{j} ,$$

where i and j label individuals in the upper and lower pools respectively. The variance is

$$\begin{matrix}Var ( Δ {p}^{*} ) = \frac{2}{{n}^{2}} {\sum }_{i , {i}^{′}} Cov ( δ {p}_{i} , δ {p}_{{i}^{′}} ) [ 1 + Cov ( δ {c}_{i}^{′} , δ {c}_{{i}^{′}}^{′} ) ] - \\ \frac{2}{{n}^{2}} {\sum }_{i , j} Cov ( δ {p}_{i} , δ {p}_{j} ) \\ = \frac{2 ( 1 - r )}{n} {σ}_{p}^{2} + \frac{2 {τ}^{2}}{n} {σ}_{p}^{2} .\end{matrix}$$

### Example 2

### Expected Allele Frequency Difference and Non-centrality Parameter

The genotype-dependent phenotype distribution is defined using a variance components model,

Xₖᵢ=Yₖ+Yₖᵢ+μₖᵢ.

Family and individual effects are normally distributed with mean zero and variance

Var(Yₖ)=t−rσ_{A}²−uσ_{D}²

Var(Yₖᵢ)=σ_{R}²−t+rσ_{A}²+uσ_{D}²

The family index is k, the sib index is i, and the individual phenotypes Xₖᵢ are the sum of Yₖ, the family effect excluding the QTL, Yₖᵢ, the individual effect excluding the QTL, and μₖᵢ, the QTL effect μ(Gₖᵢ) for sib i. The total phenotypic correlation between sibs is t. Both r and u relate to the genetic background shared between sibs, r being the genotypic correlation (1 for monozygotic twins, ½ for full sibs, ¼ for half sibs) and μ being the shared genotype expectation (1 for monozygotic twins, ¼ for full sibs, 0 for half sibs) (Falconer and Mackay 1996).

The observed phenotypes Xₖᵢ are re-expressed as family means and individual deviations from family means,

$${X}_{k •} = \frac{1}{s} {\sum }_{i} {X}_{ki}$$

$$δ {X}_{ki} = {X}_{ki} - {X}_{k •} .$$

Similar quantities are defined for the QTL effects,

$${μ}_{k •} = \frac{1}{s} {\sum }_{i} {μ}_{ki}$$

$$δ {μ}_{ki} = {μ}_{ki} - {μ}_{k •} ,$$

and the variances of the observed quantities excluding QTL effects are

$$Var ( {X}_{k •} - {μ}_{k •} ) = \frac{1}{s} [ {σ}_{R}^{2} + ( s - 1 ) ( t - r {σ}_{A}^{2} - u {σ}_{D}^{2} ) ] ≡ T {σ}_{R}^{2}$$

$$Var ( δ {X}_{ki} - δ {μ}_{ki} ) = ( 1 - T ) {σ}_{R}^{2} .$$

When the QTL effects are small, T≈(1/s)[1+(s−1)t] is an accurate approximation.

The probability that sibling 1 from family k with genotypes G=(G₁, G₂, . . . Gₛ) is selected for the upper pool is 1−Φ[X′−μ_{G})/σ], where Φ(z) is the cumulative normal probability. The variable under selection, denoted X, is either X_{k•} (between-family pools) or δXₖ₁ (within-family pools); μ_{G} is either μ_{k•} (between-family pools) or δμₖ₁ (within-family pools); the variance of X−μ_{G} is σ², either Tσ_{R}² (between-family pools) or (1−T)σ_{R}² (within-family pools); and X′ is the selection threshold applied to X. Because the labeling of sibs is arbitrary, the fraction ƒ of individuals selected for pooling is equal to the probability that sib 1 is selected, i.e. the probability that X is greater than the selection threshold,

$$f = {\sum }_{G} Pr ( G ) \{ 1 - Φ [ ( {X}^{′} - {μ}_{G} ) / σ ] \} ,$$

where Pr(G) is the probability of observing the sibship genotypes G.

To calculate the allele frequency of the selected individuals, the threshold X′ is required as a function of ƒ. Numerical inversion may be applied to the above equation. Alternatively, when the QTL effect is small (μ_{G}<σ), the linear approximation

Φ[(X′−μ_{G})/σ]≈Φ(X′/σ)−(μ_{G}/σ)φ(X′/σ)

is accurate, where φ(z)=dΦ(z)/dz is the normal probability density. The terms linear in μ_{G} cancel in the sum over G, yielding ƒ=1−Φ(X′/σ).

The expected allele frequency of the resulting pool is

$$E ( {\hat{p}}_{U} ) = \frac{1}{f} {\sum }_{G} Pr ( G ) {p}_{G} \cdot \{ 1 - Φ [ ( {X}^{′} - {μ}_{G} ) / σ ] \} ,$$

where P_{G} represents the allele frequency of sib 1. Using the linear expansion for Φ[(X′−μ_{G})/σ] yields

$$E ( {\hat{p}}_{U} ) = {\sum }_{G} Pr ( G ) {p}_{G} + \frac{φ ( {X}^{′} / σ )}{f σ} {\sum }_{G} Pr ( G ) {p}_{G} {μ}_{G} = p + \frac{φ ( {X}^{′} / σ )}{f σ} E ( {p}_{G} {μ}_{G} ) .$$

An analogous expression for the lower pools gives a symmetric result, yielding

$$E ( {\hat{p}}_{U} - {\hat{p}}_{L} ) = \frac{2 φ [ {Φ}^{- 1} ( 1 - f ) ]}{f σ} E ( {p}_{G} {μ}_{G} )$$

where X′/σ has been replaced by Φ⁻¹(1−ƒ).

The expectation of the correlation between p and μ for an individual is

$$\begin{matrix}E ( p μ ) = {p}^{2} [ a - ( p - q ) a - 2 pqd ] + 2 pq \cdot \frac{1}{2} \cdot [ d - ( p - q ) a - 2 pqd ] \\ = pq [ a - ( p - q ) d ] \\ = {σ}_{p} {σ}_{A} .\end{matrix}$$

Similarly, the correlation between sibs i and j is E(pᵢμⱼ)=rᵢⱼσₚσ_{A}, where rᵢⱼ is their genotypic correlation. Summing over sibs yields either Rσₚσ_{A} (between-family pools) or (1−R)σₚσ_{A} (within-family pools) for E(p_{G}μ_{G}), with R=(1/s)[1+(s−1)r] as before.

Selecting discordant-like sib-pairs is equivalent to selection based on |δXₖᵢ|, and the within-family analytical results are directly applicable. For larger families, discordant-like families are pre-selected in decreasing rank order of the within-family phenotypic variance

$${\sum }_{s} δ {X}_{ks}^{2}$$

summed over siblings s.

We have ascertained that the analytical results for the NCP are virtually indistinguishable from exact numerical results when the QTL effect is 5% or less of the trait variance. For larger effects, roughly when the effect size σ_{A}² approaches the minor allele frequency, the genotype-dependent phenotype distributions become resolved, transforming a complex trait into Mendelian trait amenable to traditional linkage analysis.

### Example 3

### Analytical Fit for the Optimal Pooling Fraction

Optimizing the pooling fraction is equivalent to maximizing the objective function

$$I = 2 {y}^{2} / ( f + {f}^{2} {κ}^{2} ) ,$$

where y is shorthand for φ[Φ⁻¹(1−ƒ)]. Writing ƒ as 1−Φ(z) and optimizing using dI/dz=0 yields

y·(1+2ƒκ²)−2zƒ·(1+ƒκ²)=0

We have used y=φ(z), dy/dz=−yz, and dƒ/dz=−y.

When κ is large, z is also large, and ƒ may be replaced by its asymptotic expansion for large z, ƒ=y·(z⁻¹−z⁻³). With this substitution, the optimum satisfies

$$\frac{{z}^{3}}{2 y {κ}^{2}} = 1.$$

Taking the natural logarithm of both sides and equating exponents,

$$\frac{{z}^{2}}{2} + 3 ln z - ln ( {κ}^{2} \sqrt{2 / π} ) ≡ J ( z ) = 0.$$

When κ and z are both large, the term 3ln z is asymptotically small, giving

z˜\sqrt{ln(2κ⁴/π)}≡B(κ).

An improved fit is obtained by perturbation theory by writing

z=B(κ)[1+b(κ)],

where

$${lim}_{κ \to ∞} b ( κ ) = 0.$$

Substituting this expression for z into J(z) and simplifying,

B²b+3ln[B(1+b)]=0,

which gives the asymptotic form b=(3/B²)ln B, or

z˜B−(3/B)ln B.

For clarity, the functional dependence of B and b on κ has been suppressed.

The asymptotic form provides a good fit when κ is much larger than 1 but not for smaller values. Since the asymptotic behavior for large κ is not affected by introducing terms of lower order in κ, the fit can be improved for small κ without degrading the fit at large κ by writing

z=A−(3/A)ln A+a₁,

where

$$A ( κ ) = \sqrt{{a}_{2} + ln ( 1 + {a}_{3} {κ}^{2} + \frac{2}{π} {κ}^{4} )} .$$

The constants a₁, a₂, and a₃ are then selected to fit the exact numerical results at particular values of κ. Fitting the results z=0.612 at κ=0 and z=0.8047 at κ=1 provides the particular parameters

a₁=−0.067, a₂=2, a₃=3.

TABLE I
The non-centrality parameter for family-based pooled DNA designs^{a}

| Design | F | G |
|---|---|---|
| Unrelated individuals | 1 | 1 |
| Between-family | R²/T | sR |
| Within-family | (1 − R)²/(1 − T) | 1 − r |
| Within-family, discordant | (i − r)²/2(1 − t) | 1 − r |
| pre-selection^{b} | | |
| ^{a}The non-centrality parameter (NCP) is [E(\hat{p}_{U} − \hat{p}_{L})]²/Var(\hat{p}_{U - \hat{p}}_{L}). The numerator is F.(4σ²_{A}σ²_{P}φ[Φ⁻¹(1 − f)]²/σ²_{R}f²), where F is provided for each design, f is the pooling fraction, σ²_{A} and σ²_{R} are the additive and residual variance for a QTL with allele frequency | | |
| # p, σ²_{P} is p(1 − p)/2, φ(z) is the normal probability density and Φ(z) is the cumulative normal probability. | | |
| The denominator of the NCP is [(G + τ²)σ²_{P}/Nf]+ 2ε², where G is provided for each design, τ is the coefficient of variation for DNA sample concentrations in the pool, N is the total number of individuals before selection, and ε is the raw measurement error. The combined expression for the | | |
| # NCP is (Nσ²_{A}/σ²_{R}) · [F/(G + τ²)] · {2φ[Φ⁻¹(1 − f)]²/(f + f²κ²)}, where κ² is Nε²/[(G + τ²)σ²_{P}] and κ is termed the scaled error. Each sibship has s sibs with genotypic correlation r and phenotypic correlation t; R and T are (1/s)[1 + (s − 1)r] and (1/s)[1 + (s − 1)t], respectively. | | |

^{b}Analytical results are for sib-pairs only. For larger families see numerical results (FIG. 3).

### References

Abecasis G R, Noguchi E, Heinzmann A, Traherne J A, Bhattacharyya A, Leaves N I, Anderson G G, Zhang Y, Lench N J, Carey A, Cardon L R, Moffatt M F, Cookson O C (2001) Extent and distribution of linkage disequilibrium in three genomic regions. Am J Hum Gen 68:191-197

Ardlie K G, Kruglyak L, Seielstad M (2002) Patterns of linkage disequilibrium in the human genome. Nat Rev Genet 3: 299-309

Bader J S, Bansal A, and Sham P (2001) Efficient SNP-based tests of association for quantitative phonotypes using pooled DNA. Genescreen (in press)

Barcellos L F, Klitz W, Field L L, Tobias R, Bowcock A M, Wilson R, Nelson M P, Nagatomi J, Thomson G (1997) Association mapping of disease loci, by use of a pooled DNA genomic screen. Am J Hum Gen 61:734-747

Collins F S, Guyer M S, Chakarvarti A (1997) Variations on a theme: cataloging human DNA sequence variation. Science 274:1580-1581

Daniels J, Holmans P, Williams N, Turic D, McGuffin P, Plomin R, Owen M J (1998) A simple method for analysing microsatellite allele image patterns generated from DNA pools and its applications to allelic association studies. American Journal of Human Genetics 62:1189-97

Devlin B, Roeder K (1999) Genomic control for association studies. Biometrics 55:788-808

Falconer (1965) The inheritance of liability to certain diseases estimated from the incidence among relatives. Ann Hum Gen 51: 227-33

Falconer D S, MacKay T F C (1996) Introduction to quantitative genetics. Boston: Addison-Wesley

Fisher P J, Turic D, Williams N M, McGuffin P, Asherson P, Ball D, Craig I, Eley T, Hill L, Chorney K, Chorney M J, Benbow C P, Lubinski D, Plomin R, Owen M J (1999) DNA pooling identifies QTLs on chromosome 4 for general cognitive ability in children. Hum Mol Gen 8: 915-22

Grupe A, Gerner S, Usuka J, Aud D, Belknap J K, Klein R F, Ahluwalia M K, Higuchi R, Peltz G (2001) In silico mapping of complex disease-related traits in mice. Science 292: 1915-1918

Hill, W G. (1971) Design and efficiency of selection experiments for estimating genetic parameters. Biometrics 27: 293-311

Hill L, Craig I W, Asherson P, Ball D, Eley T, Ninomiya T, Fisher P J, Turic D, McGuffin P, Owen M J, Chomey K, Chomey M J, Benbow C P, Lubinski D, Thompson L A, Plomin R (1999) DNA pooling and dense marker maps: a systematic search for genes for cognitive ability. Neuroreport 10: 843-848

Jawaid A, Bader J S, Purcell S, Cherny S S, Sham P (2002) Optimal selection strategies for QTL mapping using pooled DNA samples. European Journal of Human Genetics (in press)

Kimura M, Crow J F (1978) Effect of overall phenotypic selection on genetic change at individual loci. Proc Natl Acad Sci USA 75: 6168-6171

Ollivier L, Messer L A, Rothschild M F, Legault C (1997) The use of selection experiments for detecting quantitative trait loci. Genet Res, Camb 69: 227-232

Ott J (1999) Analysis of Human Genetic Linkage. Third edition. Johns Hopkins University Press, Baltimore

Pritchard J K, Stephens M, Rosenberg N A, Donnelly P (2000) Inference of population structure using multilocus genotype data. Genetics 155: 945-959

Pritchard J K, Rosenberg N A (1999) Use of unlinked genetic markers to detect population stratification in association studies. Am J Hum Gen 65: 220-228

Reich D E, Cargill M, Bolk S, Ireland J, Sabeti P C, Richter D J, Lavery T, Kouyoumjian R, Farhadian S F, Ward R, Lander E S (2001) Linkage disequilibrium in the human genome. Nature 411:199-204

Risch N and Teng J (1998) The relative power of family-based and case-control designs for linkage diequilibrium studies of complex human diseases I. DNA pooling. Genome Res 8:1273

Risch N, Merikangas K (1996) The future of genetic studies of complex human diseases. Science 273:1516-1517

Satten G A, Flanders D W, and Yang Q (2001) Accounting for unmeasured population substructure inb case-control studies of genetic association using a novel latent-class model. Am J Hum Gen 68: 466-477

Schork N J, Nath S K, Fallin D, Chakarvati A (2000) Linkage disequilibrium analysis of biallelic DNA markers, human quantitative trait loci, and threshold-defined case and control Subjects. Am J Hum Gen 67: 1208-1218

Sham P C, S S Cherny, S Purcell, and J K Hewitt (2000) Power of linkage versus association analyses of quantitative traits, by use of variance-components models, for sibship data. Am J Hum Gen 66: 1616-1630

Shaw S H, Carrasquillo M M, Kashuk C, Puffenberger E G, Chakravarti A (1998) Allele frequency distributions in pooled DNA samples: applications to mapping complex disease genes. Genome Res 8: 111-123

Stockton D W, Lewis R A, Abboud E B, Al Rajhi A, Jabak M, Anderson K L, Lupski J R (1998) A novel locus for Leber congenital amaurosis on chromosome 14q24. Human Genetics 103: 328-333

Suzuki K, Bustos T, Spritz R A (1998) Linkage disequilibrium mapping of the gene for Margarita Island ectodermal dysplasia (ED4) to 11q23. American Journal of Human Genetics 63:1102-1107

Zhang S, Zhao H (2001) Quantitative similarity-based association tests using population samples. American Journal of Human Genetics 69: 601-614

We claim:

1. A method for detecting an association in a population of unrelated individuals between a genetic locus and a quantitative phenotype, wherein two or more alleles occur at the locus, and wherein the phenotype is expressed using a numerical phenotypic value whose range falls within a first numerical limit and a second numerical limit, the method comprising the steps of
a) obtaining the phenotypic value for each individual in the population;
b) determining the minimum number of individuals from the population required for detecting the association using a non-centrality parameter;
c) selecting a first subpopulation of individuals having phenotypic values that are higher than a predetermined lower limit and pooling DNA from the individuals in the first subpopulation to provide an upper pool;
d) selecting a second subpopulation of individuals having phenotypic values that are lower than a predetermined upper limit and pooling DNA from the individuals in the second subpopulation to provide a lower pool;
e) for one or more genetic loci, measuring the frequency of occurrence of each allele at said locus in the upper pool and the lower pool;
f) for a particular genetic locus, measuring the difference in frequency of occurrence of a specified allele between the upper pool and the lower pool; and
g) determining that an association exists if the allele frequency difference between the pools is larger than a predetermined value.

2. The method of claim 1, wherein the difference in frequency of occurrence of the specified allele has associated with it an error of measurement.

3. The method of claim 2, wherein the error of measurement is 0.04.

4. The method of claim 2, wherein the error of measurement is 0.01.

5. The method described in claim 1, wherein the predetermined lower limit is set so that the upper pool ranges from including the highest 37% of the population to including the highest 19% of the population and the predetermined upper limit is set so that the lower pool ranges from including the lowest 37% of the population to including the lowest 19% of the population.

6. The method of claim 1, wherein the predetermined lower limit is set so that the upper pool includes the highest 27% of the population and the predetermined upper limit is set so that the lower pool includes the lowest 27% of the population.

7. The method of claim 1, wherein the genetic locus has two alleles.

8. The method of claim 1 wherein the population includes individuals who may be classified into classes.

9. The method of claim 8, wherein the classes are based on an age group, gender, race or ethnic origin.

10. The method of claim 8, wherein all the members of a class are included in the pools.

11. The method of claim 1 for determining the genetic basis of disease predisposition.

12. The method of claim 11, wherein the genetic locus which is analyzed for determining the genetic basis of disease predisposition contains a single nucleotide polymorphism.
